(19) United States
(12) Patent Application Publication
(10) Pub. No.: US 2006/0263307 A1
(43) Pub. Date: Nov. 23, 2006

(54) Packages

(75) Inventors: Joseph Edward Robillard, West Chester, OH (US); Christopher Scott Ralston, Mason, OH (US); Gary Lyle Walden, West Chester, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/435,526

(22) Filed: May 17, 2006

Related U.S. Application Data
(60) Provisional application No. 60/682,079, filed on May 18, 2005.

(51) Int. Cl. A61K 8/22

(52) U.S. Cl. 424/53; 428/551

(57) ABSTRACT

A packaged product may comprise a first package and a substance. The first package may comprise a water-impermeable film having a water vapor permeability of at least about 0.003 Grams/100 Sq In/24 hours (0.046 Grams/m^{2/}24 hours). The substance may be disposed within the first package, and may comprise a peroxide and water for tooth whitening. The first package may be disposed within a second package. The second package may comprise a water vapor controller.

### CROSS REFERENCE TO RELATED APPLICATION

This application claims the benefit under 35 USC 119(e) to U.S. application No. 60/682,079 filed May 18, 2005 which is incorporated by reference herein.

### FIELD OF THE INVENTION

Generally, packages for maintaining an active's concentration. Specifically, packages for maintaining peroxide concentration in tooth whitening products.

### BACKGROUND OF THE INVENTION

Less stable actives (including peroxides) are often incorporated into consumer products. As an example, it is desirable to include peroxides in tooth whitening products. Such incorporation of less stable actives greatly decreases the shelf life of the product. Additionally, many actives must be strictly maintained within a narrow concentration window to be safe and effective. For example, a peroxide used in a tooth whitening product may not be too concentrated as such will irritate the tissue within the mouth. However, the peroxide must be strong enough to whiten the teeth. A desirable concentration of peroxide in a tooth whitening product ranges from about 1% to about 30%.

While different packaging materials have been used to slow the loss of peroxide concentration in tooth whitening products, there is a desire to improve stability and to maintain specific concentrations of the peroxide within the tooth whitening product in order to ensure safety and efficacy and to extend shelf life of the product. Packages of the present invention can be used to stabilize peroxide concentration and increase the shelf life of these products.

Further, beyond peroxide or other traditionally unstable actives, even substances comprising traditionally stable actives may be made more stable using the packages of the present invention. Thus, the shelf life of a packaged product having traditionally longer shelf life may be extended. There is an opportunity for improvement of many packaged products comprising an active and a solvent, including packaged personal health care products. Packages of the present invention can be used to stabilize peroxide concentration and increase the shelf life of these products also.

Additionally, beyond stabilizing the substances comprising an active and a solvent, packages of the present invention may be used to decrease or eliminate bloating (that is, puffing up) of packages. The vapor that often builds within a package comprising a solvent may be transmitted through packages of the present invention, such that bloating does not occur.

### SUMMARY

A packaged product may comprise a package and a substance. The package may comprise a water-impermeable film having a water vapor permeability of at least about 0.003 Grams/100 Sq In/24 hours (0.046 Grams/m²/24 hours), at 90° F. (32° C.) and 100% RH. The substance may be disposed within the package, and may comprise a peroxide and water.

A package system may comprise a first package, a substance, and a second package. The first package may comprise a water-impermeable film having a water vapor permeability of at least about 0.003 Grams/100 Sq In/24 hours (0.046 Grams/m²/24 hours), at 90° F. (32° C.) and 100% RH. The substance may be disposed within the first package, and may comprise a peroxide. The second package may enclose the first package and may comprise a water vapor controller.

### BRIEF DESCRIPTION OF THE DRAWINGS

FIG. 1 is an exploded cross-sectional side view of a first package.

FIG. 2 is an exploded cross-sectional side view of a first package.

FIG. 3 is an exploded cross-sectional side view of a substance.

FIG. 4 is a cross-sectional top view of a second package.

### DETAILED DESCRIPTION

Generally, substances comprising peroxides in a solvent (such as water, ethanol, ethyl acetate, silicones, flavors, etc.) may be used for whitening the teeth. These substances may be contained within a first package. The first package may comprise one or more layers which allow the entry or escape of solvent vapor for stabilizing the concentration of the peroxide over a predetermined amount of time. The first package may comprise a metal layer.

As the first package may create a first stabilizing environment for the peroxide containing substance, a second package may further be used to create a second stabilizing environment. The second package may be used to draw or drive solvent vapor from or into the first package for maintaining a more constant and more stable peroxide concentration over time. Beyond stabilization, the second package may create an environment within the first package which keeps the peroxide containing substance adequately hydrated for immediate use upon removal from the first package.

### I. DEFINITIONS

The following definitions are listed alphabetically:

As used herein, the phrase “air-impermeable” is intended to refer to a barrier where the transmission of oxygen is less than 0.01 cubic centimeters (cc)/1 square inch (sq In)/30 days (15.5 cc/m²/24 hours) @ 73 degrees Fahrenheit (° F.) (23° C.) @ 0% relative humidity (RH). See ASTM D-3985, Oxygen Transmission Rate.

As used herein, the term “peroxide” (and its derivatives) is intended to refer to compounds that generate hydrogen peroxide when contacted with an aqueous media. Examples of a peroxide include, but are not limited to, hydrogen peroxide, carbamide peroxide, sodium percarbonate, etc.

As used herein, the phrase “peroxide concentration” is intended to refer to the equivalent concentration of hydrogen peroxide created from any peroxide generating species, expressed as a weight percentage.

As used herein, the phrase “peroxide degradation rate” (PDR) is intended to refer to the rate at which peroxide concentration is reduced in a product. Factors which may contribute to peroxide decomposition include: auto-oxidation, increasing temperature (approximately a 2.2 factor increase for each 10 degrees Celsius (° C.)); increasing pH (especially at pH about >6-8); increasing contamination (especially transition metals such as copper, manganese, or iron); and to a lesser degree, exposure to ultraviolet light.

The PDR is defined herein as the change of peroxide concentration over time:
PDR=([Initial peroxide concentration]−[peroxide concentration at time from beginning of experiment (t)])/t
PDR is measured as a function of temperature, humidity, and product matrix composition, or combinations thereof. Typical storage condition guidelines are set out by the International Committee on Harmonization (ICH). Peroxide concentration is typically measured by using peroxide sensitive tests, including indirect iodometric titration, permanganate titration, and other techniques well known in the art.

As used herein, the phrase “water-impermeable” is intended to refer to a barrier where the transmission of liquid water is less than 0.1 g/100 sq In/month (1.55 Grams/m²/month)@ 25° C. @ 60% RH as measured by the ASTM F-1249, TAPPI T557, JIS K-7129).

As used herein, the phrase “water vapor controller” is intended to refer to a system which controls the concentration of water vapor within a product to a desired level over the life of the product. The system may utilize one or more of packaging designs, materials, humectants, or desiccants.

As used herein, the phrase “water vapor permeable” is intended to refer to a barrier where the transmission of water vapor is equal to or greater than 0.001 g/100 Sq In/day (0.0155 Grams/m²/day) at 90° F. (30° C.) and 100% RH (ASTM F-1249, TAPPI T557, JIS K-7129).

### II. FIRST PACKAGE

In one embodiment a first package 10 may be constructed of a water-impermeable film or sheet comprised of multiple layers, including, but not limited to, one or more plastic layer(s), one or more metal layer(s), one or more glass layer(s), and/or one or more silicone oxide layer(s). Layers may act as barriers and/or may act to tie together other layers to form the film or sheet. For example one or more layers can comprise adhesive layer. The film or sheet of the first package 10 may have a thickness from about 1 mil to about 3 mils (about 25.4 to about 76.2 microns), from about 1.1 mils to about 2.5 mils (about 27.94 to about 63.5 microns), from about 1.4 mils to about 2.2 mils (about 35.56 to about 55.88 microns), or from about 1.6 mils to about 1.8 mils (about 40.64 to about 45.72 microns).

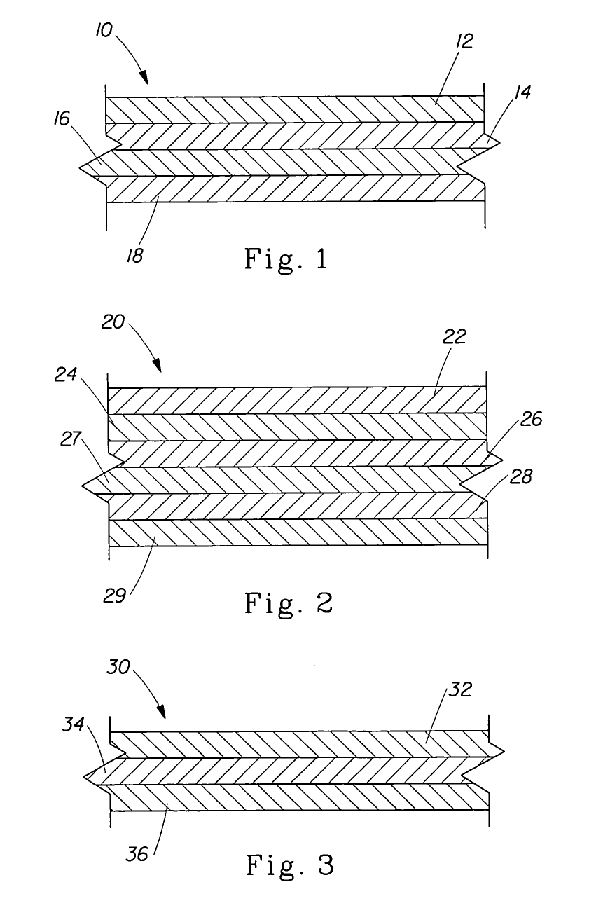

As shown in FIG. 1, the first package may be comprised of four layers. A first package layer 12 may comprise a sealable plastic. The first package layer 12 may more specifically comprise one or a combination of polypropylene, polyethylene, cellophane, oriented polypropylene, Surlyn® manufactured by DuPont, polyethylene terephthalate, and glass. This layer may be in contact with a tooth whitening product. This layer may be clear.

A second package layer 14 may comprise a metal. The second package layer may more specifically comprise one or a combination of aluminum, gold, silver, platinum, tin, nickel, and copper. The metal of the second layer 14 may have a thickness from about 50 angstroms (Å) to about 360 Å, from about 70 Å to about 340 Å, from about 100 Å to about 320 Å, or from about 120 Å to about 320 Å.

A third package layer 16 may comprise an adhesive. The third package layer 16 may more specifically comprise one or a combination of polyethylene, ethyl vinyl acetate, ethylene acid copolymer, ethyl vinyl alcohol, polyvinyl acetate, polyurethane, acrylic, vinyl, and ethylene acrylic acid (EAA), etc.

A fourth package layer 18 may comprise a plastic. The fourth package layer 18 may more specifically comprise one or a combination of polyethylene terephthalate, polypropylene, polyethylene, or nylon. This layer may have ink printing on either side.

A third package layer 16 may not be necessary. For instance, the fourth package layer 18 comprising polypropylene or polyethylene may be directly bonded with the second package layer 14 (the second package layer acting as a tie layer as described above).

The first package layer 12 may have the second package layer 14 applied to it (e.g., via vacuum depositing, sputtering, vacuum evaporation, or electroplating; See, for example, Fundamentals of Packing Technology, Soroka, Walter, Institute of Packing Professionals (1999), and The Wiley Encyclopedia of Packaging Technology, Bakker, Marilyn (1986)). The third package layer 16 may be used to tie or adhere the fourth package layer 18 to the second package layer 14. For example, a layer of polypropylene may have a layer of aluminum vacuum deposited onto it. A third package layer 16, such as ethyl vinyl acetate (EVA), may be used to adhere a layer of polyethylene to the aluminum. Section VI (below) may be referred to for a more specific example of how a first package may be formed.

The first package 10 and/or the water-impermeable film of the first package 10 may have a water vapor permeability, from about 0.003 Grams/100 Sq In/24 hour (0.0465 Grams/m²/24 hour) to about 0.07 Grams/100 Sq In/24 hour (1.085 Grams/m2/24 hour), from about 0.003 Grams/100 Sq In/24 hour to about 0.03 Grams/100 Sq In/24 hour (0.0465 Grams/m²/24 hour), or from about 0.003 Grams/100 Sq In/24 hour (0.0465 Grams/m²/24 hour) to about 0.02 Grams/100 Sq In/24 hour (0.31 Grams/m²/24 hour), as measured at 90° F. (32° C.) and 100% RH (ASTM F1249). Water vapor permeability may be impacted by the metal layer. The metal layer can be used as the main barrier to water vapor of the first package 10. Variances of this layer may significantly impact the water vapor permeability of the first package 10.

In another embodiment, as shown in FIG. 2, the first package 20 may be comprised of six layers. A first package layer 22 may comprise a sealable plastic. The first package layer 22 may more specifically comprise one or a combination of polypropylene, polyethylene, cellophane, oriented polypropylene, Surlyn® manufactured by DuPont, polyethylene terephthalate, and glass. This layer may be in contact with a tooth whitening product.

A second package layer 24 may comprise an adhesive. The second package layer 24 may more specifically comprise one or a combination of polyethylene, ethyl vinyl acetate, ethylene acid copolymer, ethyl vinyl alcohol, polyvinyl acetate, polyurethane, acrylic, vinyl, and ethylene acrylic acid (EAA), etc.

A third package layer 26 may comprise a plastic. The third package layer 26 may more specifically comprise one or a combination of polypropylene, polyethylene, cellophane, oriented polypropylene, Surlyn® manufactured by DuPont, polyethylene terephthalate, and glass.

A fourth package layer 27 may comprise a metal. The fourth package layer may more specifically comprise one or a combination of aluminum, gold, silver, platinum, tin, nickel, and copper. The metal of the fourth layer 27 may have a thickness from about 50 angstroms (Å) to about 360 Å, from about 70 Å to about 340 Å, from about 100 Å to about 320 Å, or from about 120 Å to about 320 Å.

A fifth package layer 28 may comprise an adhesive. The fifth package layer 28 may more specifically comprise one or a combination of polyethylene, ethyl vinyl acetate, ethylene acid copolymer, ethyl vinyl alcohol, polyvinyl acetate, polyurethane, acrylic, vinyl, and ethylene acrylic acid (EAA), etc.

A sixth package layer 29 may comprise a plastic. The sixth package layer 29 may more specifically comprise one or a combination of polyethylene terephthalate, polypropylene, polyethylene, or nylon. This layer may have ink printing on either side.

A fifth package layer 28 may not be necessary. For instance, the fifth package layer 28 comprising polypropylene or polyethylene may be directly bonded with the fourth package layer 27 (the fourth package layer acting as a tie layer as described above).

### III. SUBSTANCE

The substance 30 may comprise multiple layers, including, but not limited to, a plastic layer and/or a gel layer. In one embodiment, as shown in FIG. 3, the substance 30 may be comprised of three layers. A first substance layer 32 may comprise a plastic. The first substance layer 32 may specifically comprise one or a combination of polypropylene or polyethylene.

A second substance layer 34 may comprise a gel. The second substance layer 34 may more specifically comprise one or a combination of hydrogels, such as polyacrylic acid, methyl cellulose, poloxamer, polyethylene oxide, or polyvinyl alcohol. The second substance layer may additionally comprise one or a combination of actives, such as a peroxide, a fluoride, or an antimicrobial. Further, the second substance layer 34 may comprise a solvent. The solvent may be one or a combination of water, ethanol, ethyl acetate, silicones, flavors, etc. Peroxide concentration may be from about 0.1% to about 30%, from about 1% to about 7%, from about 5% to about 11%, or from about 6% to about 16%. The second substance gel may be referred to as a “peroxide substrate”. A third substance layer 36 may comprise a plastic. The third substance layer may more specifically comprise one or a combination of polypropylene, polyethylene, or polyethylene terephthalate.

### IV. PACKAGED SUBSTANCE

The substance 30 may be contained within the first package 10, 20 (said combination herein, “a packaged substance” 37). A packaged substance 37, wherein the substance 30 comprises a peroxide, may have a PDR from about 0.3% to about 8% per 4 months, from about 0.6% to about 7% per 4 months, or from about 1% to about 6% per 4 months (measured at 40° C. and at 75% RH). Thus, a packaged substance 37 comprising a peroxide may maintain a peroxide concentration from about 0.1% to about 30%, from about 1% to about 7%, from about 5% to about 11%, or from about 6% to about 16% for from about 6 months to about 4 years, from about 9 months to about 3 years, or from about 1 year to about 2 years (measured at 25° C. and 60% RH). From about 1 to about 200 individual packaged substances can be contained within a second package. In another embodiment from about 10 to about 90 individual packaged substances are placed into a second package. In yet another embodiment from about 20 to about 50 individual packaged substances are placed into a second package. In yet another embodiment from about 70 to about 90 individual packaged substances are placed into a second package, and in yet another embodiment from about 1 to about 10 individual packaged substances are placed into a second package.

### V. PACKAGE SYSTEM

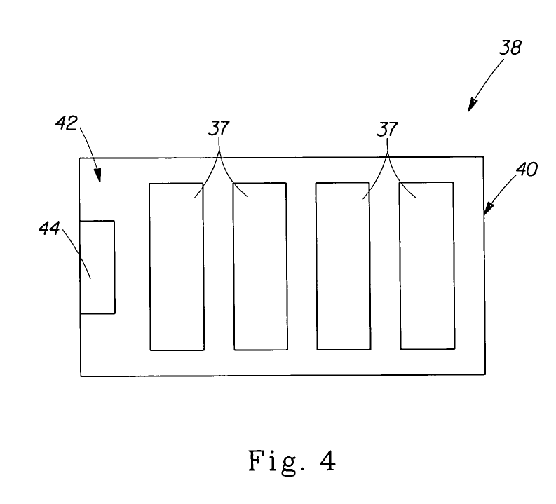

A packaged substance 37 may be contained within a second package 40 (said combination herein, a “package system” 38) as shown in FIG. 4. The second package 40 may form an additional, second barrier (that is, beyond the first package 10, 20) between the environment and the substance 30. Specifically, the first package 10, 20 creates a first environment for the substance 30, and the second package 40 creates a second environment for the substance 30. The environment of the second package 40 may be used to impact the RH of the environment of the first package 10, 20. Package system 38 may be ideal for geographies which are more extremely dry or humid.

As shown in FIG. 4, the package system 38 may comprise an interior space 42 controlled by a water vapor controller 44. The water vapor controller 44 may be a humidifier, a desiccant, or combination of both. The humidifier may be one or a combination of aqueous solutions of glycerin, sorbitol, PEG, water etc. The desiccant may be one or a combination of silica, magnesium sulfate, potassium carbonate, etc. Alternatively, the water vapor controller 44 may be a material that acts as both a humectant and a desiccant, depending on the condition (for example, the materials and systems disclosed in U.S. Pat. No. 5,936,178, filed Jun. 10, 1997; and U.S. Pat. No. 6,244,432, filed Aug. 9, 1999). In the case where the substance comprises a solvent other than water, including, but not limited to, ethanol, ethyl acetate, silicones, and flavors, different solvent vapor controllers may be used, including, but not limited to, activated carbon, zeolites, cyclodextrins, and molecular sieves. Additionally, a relative humidity may be maintained within the first package 10, 20, without the use of a water vapor controller, by placing the first package 10, 20 into the second package 40 and sealing the second package 40 closed. The space 42 in the second package 40 surrounding the first package 10, 20 can fill with the water vapor which permeates through the first package 10, 20 into the space 42 of the second package 40. The two packages can reach a state of equilibrium and therefore control the amount of water vapor that can permeate from the first package.

The package system 38, can have an internal controlled RH of about 60% (measured at 25° C.). The package system maintains the 60% RH within the packaged substance regardless of the conditions external to the second package.

The package system can have a RH of from about 40% to about 75% (measured at 25° C.). In another embodiment the RH of the package system can be from about 45% to about 55%, and in yet another embodiment the RH of the package system can be about 50% to about 60% (measured at 25° C.). Maintaining this RH can stabilize the amount of water loss from the packaged substance, and therefore stabilizing the environment of the package.

### VI. EXAMPLES

A. First Package
In one embodiment a first package 10 is formed using a metallized foil film or sheet which is produced using a 300 angstroms (Å) aluminum film deposited on 60 gauge (ga) oriented polypropylene (OPP), laminated with 8 lb low density polyethylene (LDPE) to a 48 ga polyethylene terephthalate (PET) exterior plastic.
The film or sheet of the first package 10 is utilized in two sections, top and bottom. A substance 30 is registered on the bottom section of the first package 10. The top section of the first package 10 is applied to the substance 30 and the bottom section of the first package 30, and is registered and heat sealed on all sides. This structure is then slit and cross cut into individual pouch containment systems.

Additional examples of metallized films or sheet are disclosed in the table below:

TABLE 1
Examples of Foil Composition

| | | | |
|---|---|---|---|
| Exterior | 92 ga (23.37 | 92 ga (23.37 microns) | 48 ga (12.2 microns) |
| | microns) | PET | PET |
| | PET | | |
| Adhesive | 5 ga (1.27 | 5 ga (1.27 microns) | 56 ga (14.22 microns) |
| | microns) | EVA | 8 lb LDPE |
| | EVOH | | |
| Interior | 60 ga (15.24 | 60 ga (15.24 microns) | 60 ga (15.24 microns) |
| | microns) | Metallized OPP | Metallized OPP |
| | Metallized | | |
| | OPP | | |

B. First Package
In another embodiment a first package 20 is formed using a layer of 70 gauge oriented polypropylene (OPP) that is adhered to a metallized foil film or sheet which is produced using a 300 angstroms (Å) aluminum film deposited on 55 gauge (ga) oriented polypropylene (OPP), laminated with 8 lb low density polyethylene (LDPE) to a 92 ga polyethylene terephthalate (PET) exterior plastic.
The film or sheet of the first package 20 is utilized in three sections, top, middle and bottom. A substance 30 is registered on the bottom section of the first package 20. The top section of the first package 20 is applied to the substance 30 and the bottom section of the first package 30, and is registered and heat sealed on all sides. This structure is then slit and cross cut into individual pouch containment systems.

Additional examples of metallized films or sheet are disclosed in the table below:

TABLE 2
Examples of Foil Composition

| | | | |
|---|---|---|---|
| Exterior | 92 ga (23.37 | 92 ga (23.37 microns) | 48 ga (12.2 microns) |
| | microns) | PET | PET |
| | PET | | |
| Adhesive | 5 ga (1.27 | 5 ga (1.27 microns) | 56 ga (14.22 microns) |
| | microns) | EVA | 8 lb LDPE |
| | EVOH | | |
| Center | 55 ga (15.24 | 60 ga (15.24 microns) | 60 ga (15.24 microns) |
| | microns) | Metallized OPP | Metallized OPP |
| | Metallized | | |
| | OPP | | |
| Adhesive | 5 ga (1.27 | 5 ga (1.27 microns) | 56 ga (14.22 microns) |
| | microns) | EVA | 8 lb LDPE |
| | EVOH | | |
| Interior | 70 ga (15.24 | 60 ga (15.24 microns) | 60 ga (15.24 microns) |
| | microns) | OPP | OPP |
| | OPP | | |

B. Substance
Examples of a substance 30 are described in U.S. Pat. No. 6,136,297, filed Mar. 17, 1998; U.S. Pat. No. 6,096,328, filed Nov. 19, 1998; U.S. Pat. No. 6,045,811, filed Jun. 6, 1997; U.S. Pat. No. 5,989,569, filed Jun. 6, 1997; U.S. Pat. No. 5,894,017, filed Jun. 6, 1997; U.S. Pat. No. 5,891,453, filed Mar. 17, 1998; U.S. Pat. No. 5,879,691, filed Jun. 6, 1997; U.S. Pat. No. 6,277,458, filed Mar. 15, 1999; U.S. Pat. No. 6,461,158, filed Aug. 14, 2000; and U.S. Pat. No. 6,551,579, filed May 29, 2001; And US Pub. Nos. 2003/0211056, filed Apr. 9, 2003; and 2004/0120903, Sep. 10, 2003. These same references also include examples of a first package 20 not comprising a water vapor permeable layer.
Additional examples of substances 30 and first packages 10, 20 may be described in U.S. Pat. No. 5,376,006, filed Sep. 24, 1992; U.S. Pat. No. 5,409,631, filed Nov. 22, 1991; U.S. Pat. No. 5,746,598, filed Sep. 27, 1996; U.S. Pat. No. 5,770,105, filed Sep. 30, 1996; U.S. Pat. No. 6,730,316, filed Nov. 5, 2002; U.S. Pat. No. 6,500,408, filed Jan. 27, 2001; U.S. Pat. No. 6,503,486, filed Feb. 22, 2002; U.S. Pat. No. 6,514,483, filed Feb. 22, 2002; U.S. Pat. No. 6,419,906, filed Mar. 12, 2001; U.S. Pat. No. 6,669,930, filed Jan. 15, 2003; U.S. Pat. No. 6,770,266, filed May 24, 2002; U.S. Pat. No. 5,851,551, filed Jun. 21, 1994; U.S. Pat. No. 6,689,344, filed Sep. 13, 2002; U.S. Pat. No. 6,682,721, PCT filed Feb. 13, 2001; U.S. Pat. No. 5,922,307, filed Sep. 25, 1996; U.S. Pat. No. 6,331,292, filed Nov. 16, 1998; U.S. Pat. No. 6,488,914, filed Oct. 31, 2001; U.S. Pat. No. 6,517,350, filed Mar. 5, 2001; U.S. Pat. No. 5,700,478, PCT filed Aug. 19, 1994, U.S. Pat. No. 6,210,699, filed Apr. 1, 1999; U.S. Pat. No. 5,948,430, filed Aug. 1, 1997, U.S. Pat. No. 6,709,671, filed May 14, 2002; U.S. Pat. No. 6,284,264, filed Aug. 2, 2000; U.S. Pat. No. 6,177,096, filed Apr. 6, 1999; and U.S. Pat. No. 5,948,430, filed Aug. 1, 1997; And US App. Nos. 2003/0152528, filed Feb. 5, 2003; 2003/0170308, filed May 1, 2002; 2004/0105834, filed Sep. 12, 2003; and 2004/0062724, filed May 23, 2003.

C. Second Package
A water vapor controller 44 (e.g., Humidipak®, from Humidipak, Inc.) is fixed to the inside portion of a second package 40 (made of cardboard or OPP plastic) and from about 1 to about 200 individual packaged substances 37 are placed into the second package 40. The second package 40 is closed and sealed using shrink wrap, for shipping and pre-use by the consumer.

### VII. TEST PROCEDURES

A. Peroxide Analysis

The level of hydrogen peroxide in a substance is determined using an indirect iodometric titration analysis on a substance having three layers. The first layer is a base plastic layer, the second layer is a gel layer comprising hydrogen peroxide, and the third layer is a plastic layer covering the second layer. The third layer may be peeled away from the first layer such that a substantial portion of the second gel layer is present thereon.

Peel the third layer comprising the second layer from the first layer and place in a tared 250 mL beaker. Record the total sample weight to the nearest 0.0001 g. Add a magnetic stirbar and 100 mL of 0.04 N sulfuric acid and cover with parafilm. Stir for a minimum of 10 minutes, or until second layer separates from the third layer and is visually dispersed. Add 25 mL of 10% potassium iodide solution and 3 drops of 10% ammonium molybdate solution, cover and stir for an additional 3 minutes. Fill a 50 mL buret with standardized 0.03 N sodium thiosulfate and titrate slowly but steadily with constant agitation. Titrate to a pale, straw-yellow color. Add about 1 mL of the starch indicator solution and continue the titration dropwise, with agitation, until solution is colorless. Record mL of titrant used to reach endpoint. After the titration, remove the third layer from the titration beaker, rinse with water and blot dry with a laboratory wipe. Weigh the contents and record to the nearest 0.0001 g. The sample weight is determined by subtracting the weight of the third layer from the pre-test weight of the combined second and third layers. The percentage hydrogen peroxide (% H₂O₂) is calculated using the following calculation:

$$\% {H}_{2} {O}_{2} = \frac{( Thiosulfate Used ( mL ) \times Thiosulfate Normality ( meq / mL ) \times 17.01 ( mg / meq ) \times 100 )}{Pre \text{-} test Substance Wt . ( g ) \times 1000 ( mg / g )}$$

B. Water Vapor Permeability

A dry chamber is separated from a wet chamber of known temperature and humidity by the barrier material to be tested. The dry chamber and the wet chamber make up a diffusion cell in which the first package is sealed. Water vapor diffusing through a sample of the first package mixes with the gas in the dry chamber and is carried to a pressure-modulated infrared sensor. This sensor measures the fraction of infrared energy absorbed by the water vapor and produces an electrical signal, the amplitude of which is proportional to water vapor concentration. The amplitude of the electrical signal produced by the test film is then compared to the signal produced by measurement of a calibration film of known water vapor transmission rate. This information is then used to calculate the rate at which moisture is transmitted through the sample of first package being tested. See ASTM F 1249-01, Water Vapor Transmission Rate Through Plastic Film and Sheeting Using a Modulated Infrared Sensor.

The present invention may additionally include information that will communicate to the consumer, by words and/or by pictures, that use of the invention will provide benefits associated with the first package, substance, second package, packaged substance, and/or the package system. This information may include a claim of superiority over other like products. Accordingly, the use of packages in association with information will communicate to the consumer, by words and or by pictures, that use of the invention will provide the particular and related benefits as previously mentioned above. The information may include, for example, advertising in all of the usual material, as well as statements and icons on the package, or elements of the first package, second package, packaged substance, and/or the package system, to inform the consumer.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A packaged substance for whitening teeth, comprising:
a package comprising a water-impermeable film, said water-impermeable film having a water vapor permeability of at least about 0.003 Grams/100 Sq In/24 hours at 32° C. and 100% relative humidity; and
a substance disposed within said package, wherein said substance comprises a peroxide and water, wherein said substance is for whitening teeth.

2. The packaged substance of claim 1, wherein said water-impermeable film has a water vapor permeability from about 0.003 Grams/100 Sq In/24 hours to about 0.07 Grams/100 Sq In/24 hours 32° C. and 100% relative humidity.

3. The packaged substance of claim 1, wherein the concentration of said peroxide is at least about 0.1% by weight of said substance.

4. The packaged substance of claim 1, wherein said substance has a peroxide degradation rate of about 1% over 4 months measured at 40° C. and 75% relative humidity.

5. The packaged substance of claim 1, wherein said substance has a peroxide degradation rate from about 1% to about 6% over 4 months measured at 40° C. and 75% relative humidity.

6. The packaged substance of claim 1, wherein said water-impermeable film is selected from the group consisting of a metal layer, a glass layer, a plastic layer, and a silicone oxide layer.

7. The packaged substance of claim 6, wherein said metal layer has a thickness from about 50 Å to about 300 Å.

8. The packaged substance of claim 7, wherein said metal layer is selected from the group consisting of, aluminum, gold, silver, platinum, tin, nickel, and copper.

9. The packaged substance of claim 1, wherein water-impermeable film comprises a first layer and second layer.

10. The packaged substance of claim 9, wherein said second layer is selected from the group consisting of, aluminum, gold, silver, platinum, tin, nickel, and copper.

11. The packaged substance of claim 9, wherein said first layer is selected from the group consisting of polypropylene, polyethylene, Teflon®, cellophane, and oriented polypropylene.

12. A package system, comprising:
a first package comprising a metal layer, said metal layer having a thickness from about 50 Å to about 360 Å;
a substance disposed within said first package, wherein said substance comprises a peroxide, wherein said substance is for whitening teeth;
a second package enclosing said first package; and
wherein said second package comprises a water vapor controller.

13. The package system of claim 12, wherein said water vapor controller is a humidifier.

14. The package system of claim 13, wherein said humidifier is water.

15. The package system of claim 12, wherein said second package is sealed around said first package.

16. The package system of claim 12, wherein said metal layer comprises aluminum.

17. The package system of claim 12, wherein said package system has a relative humidity of from about 40 to about 60% measured at 25° C.

18. A packaged substance for whitening teeth, comprising:
a package comprising a plurality of layers, one of said layers being a metal layer, said metal layer comprising aluminum having a thickness of from about 50 Å to about 360 Å;
a substance disposed within said package, wherein said substance comprises a gel, wherein said gel comprises a peroxide and a solvent, wherein said peroxide has a peroxide concentration from about 2% to about 30%, wherein said substance is for whitening teeth; and
wherein said package allows for the transmission of solvent vapor through it.

19. The packaged substance of claim 18, wherein said package allows for the transmission of solvent vapor through it at a rate of from about 0.003 Grams/100 Sq In/24 hours to about 0.07 Grams/100 Sq In/24 hours 32° C. and 100% relative humidity.

20. The packaged substance of claim 19, wherein said solvent comprises water.
